(19) United States
(12) Patent Application Publication
(10) Pub. No.: US 2004/0193253 A1
(43) Pub. Date: Sep. 30, 2004

(54) Replacement venous valve

(76) Inventors: Patricia E Thorpe, Iowa City, IA (US); Francisco J Osse, Sao Paulo (BR)

Correspondence Address: INTELLECTUAL PROPERTY GROUP, FREDRIKSON & BYRON, P.A., 200 SOUTH SIXTH STREET, SUITE 4000, MINNEAPOLIS, MN, 55402, US

(21) Appl. No.: 10/476,363

(22) PCT Filed: Apr. 30, 2002
(86) PCT No.: PCT/US02/13640

Related U.S. Application Data
(62) Division of application No. 60/287,519, filed on Apr. 30, 2001.
(62) Division of application No. 60/312,814, filed on Aug. 16, 2001.

(51) Int. Cl. A61F 2/06

(52) U.S. Cl. 623/1.24; 623/2.14

(57) ABSTRACT

A replacement venous valve comprises a pair of support wings and a pair of valve wings. The valve wings are designed to deploy first from a catheter deployment device and provide stability while the support wings then deploy. The valve wings support the venous valve material and the support wings maintain patency of the vein above the valve while simultaneously anchoring the location and orientation of the valve.

### FIELD OF THE INVENTION

The present invention relates to venous valve replacement and, in particular, to replacement venous valves to lower extremities and a therapeutic method of treating venous circulatory disorders.

### BACKGROUND OF THE INVENTION

Chronic venous insufficiency (CVI) of the lower extremities is a common condition that is considered a serious public health and socioeconomic problem. In the United States, approximately two million workdays are lost each year, and over 2 million new cases of venous. thrombosis are recorded each year. About 800,000 new cases of venous insufficiency syndrome will also be recorded annually. Ambulatory care costs of about $2,000, per patient, per month, contribute to the estimated U.S. cost of $16,000,000 per month for the treatment of venous stasis ulcers related to CVI.

It is estimated that greater than 3% of the Medicare population is afflicted by a degree of CVI manifested as non-healing ulcers. Studies have indicated that about 40% of seriously affected individuals cannot work or even leave the house except to obtain medical care: It is estimated that 0.2% of the American work force is afflicted with CVI.

Chronic venous insufficiency arises from long duration venous hypertension caused by valvular insufficiency and/or venous obstruction secondary to venous thrombosis. Other primary causes of CVI include varicosities of long duration, venous hypoplasia and arteriovenous fistula. The signs and symptoms of CVI have been used to classify the degree of severity of the disease, and reporting standards have been published. Studies demonstrate that deterioration of venous hemodynamic status correlates with disease severity. Venous reflux, measured by ultrasound studies, is the method of choice of initial evaluation of patients with pain and/or swelling in the lower extremities. In most serious cases of CVI, venous stasis ulcers are indicative of incompetent venous valves in all systems, including superficial, common, deep and communicating veins. This global involvement affects at least 30% of all cases. Standard principles of treatment are directed at elimination of venous reflux. Based on this observation, therapeutic intervention is best determined by evaluating the extent of valvular incompetence, and the anatomical distribubon of reflux. Valvular incompetence, a major component of venous hypertension, is present in about 60% of patients with a clinical diagnosis of CVI.

Endovascular valve replacement refers to a new concept and new technology in the treatment of valvular reflux. The concept involves percutaneous insertion of the prosthetic device under fluoroscopic guidance. The device can be advanced to the desired intravascular location using guide wires and catheters. Deployment at a selected site can be accomplished to correct valvular incompetence. Percutaneous placement of a new valve apparatus provides a less invasive solution compared to surgical transposition or open repair of a valve.

The modern concept of a stent was introduced in the 1960s. Subsequently, it has been successfully incorporated in the treatment of arterioral aneurysms and occlusive disease. The use of endovascular stents represents one of the most significant changes in the field of vascular surgery since the introduction of surgical graft techniques in the early 1950s.

Initially, the dominant interest of vascular specialists was application of stents in the arterial system. The venous system and venous disease were not considered an arena for stent application. The utilization of endovascular treatment in venous disease was initially confined to the treatment of obstruction, in the pelvic veins (for CVI) as well as treatment of obstructed hemodialysis access grafts and decompression of portal hypertension (TIPS). Although these procedures enjoy widespread application, the actual number of patients involved is relatively low compared to the number afflicted with CVI and related syndrome. Thus, the necessity for therapy using endovascular technology for the treatment of venous disease arose. The prevalence of CVI and the magnitude of its impact demand development of an effective alternative therapy.

### BRIEF SUMMARY OF THE INVENTION

A replacement venous valve comprises a pair of support wings and a pair of valve wings. The valve wings are designed to deploy first from a catheter deployment device and provide stability while the support wings then deploy. The valve wings support the venous valve material and the support wings maintain patency of the vein above the valve while simultaneously anchoring the location and orientation of the valve.

### BRIEF DESCRIPTION OF THE DRAWINGS

FIG. 1 is a schematic depiction of the frame of one embodiment of a replacement valve.

FIG. 2 is a generally top perspective view of one embodiment of the replacement valve.

FIG. 3 is a schematic side section view of a replacement valve in a vein.

FIG. 4 is a schematic side view of one embodiment of the angular relationship of a replacement valve structure in a vein.

FIG. 5 is a side section view of a compressed, non-deployed replacement valve in a delivery system component.

FIG. 6 is a schematic side section view of a venous valve being localized prior to release in a vein.

FIG. 7 is a schematic side section view of the valve wing release opening of a venous valve in a vein.

FIG. 8 is a schematic side section view of the stabilizer wing release of a venous valve in a vein.

FIG. 9 is a schematic side section view of a venous valve being localized prior to release in a vein.

FIG. 10 is a schematic side section view of the valve wing release of a venous valve in a vein.

FIG. 11 is a schematic side section view of the valve wing positioning and release of the stabilizer wing in a venous valve in a vein.

FIG. 12 is a schematic side section view of the valve wing positioned and the stabilizer wing being deployed in a venous valve in a vein.

FIG. 13 is a schematic side section view of the valve wing and stabilizer wing fully deployed in a vein.

FIG. 14 is a schematic side section view of the valve functioning in position in a vein.

FIG. 15 is an assembly view of an alternate embodiment replacement valve design.

### DETAILED DESCRIPTION OF THE INVENTION

Within the field of endovascular treatment, no previous technology has effectively used a replacement valve which also acts similar to a self-righting stent in a percutaneously located assembly. Indeed, recognition of the need for such a device, system and method of employment has been lacking. Attempts at venous valve repair are not common. Indeed, minimally invasive repair or replacement procedures are quite uncommon. This is due, in part, to the poor availability of properly sized and properly designed prosthetic venous valves. U.S. Pat. No. 5,500,014 has an excellent discussion of the different attempts to provide prosthetic venous valves, and such discussion is incorporated by reference herein. For the anatomy of venous valves, an excellent reference includes Venous Valves, by R. Gottlub and R. May, published by Springer Verlag, Austria, 1986.

The inventors have devised a device, system and method of deployment for a valve assembly utilizing various materials having excellent cost, biocompatibility, and ease of use. In one embodiment, a stent is assembled having excellent length and stability characteristics, as well as an improved profile for ease of placement and automatic deployment at a deployment site. The assembly does not rely only on placement at a previous valvular site but may also be utilized either proximal or distal to the incompetent valve site due to the self-expanding and self-orienting features and improved anti-migration characteristics of the assembly.

The use of the material chosen for endovascular valve leaflet portions of the replacement valve of this assembly may be selected from a variety of biocompatible substances Whether the material is formed of elastomer, sclera, small intestine sub-mucosa (SIS), other mammalian tissue, or other suitable material, the venous stent device of this invention may serve as a substitute for deteriorated venous valves which have been altered by thrombosis or congenital hypoplasia. The valve prosthesis which self-expands similar to a stent will be percutaneously introduced with a small sized catheter delivery system, but demonstrates improved self-righting and orienting within the vein.

Justification for development of this invention is based on the incidence of venous disorders that lack adequate endovascular therapy. Patients who are treated surgically undergo a more invasive method that involves greater costs and more numerous potential complications. The minimally invasive technique of this invention will decrease length of hospital stay, lower over-all costs and permit an almost immediate return to normal activity. Indeed, it is believed that the availability of this treatment will dramatically alter the lives of many people, including those who might not have been able to undergo previous surgical techniques for the repair or replacement of damaged venous valves.

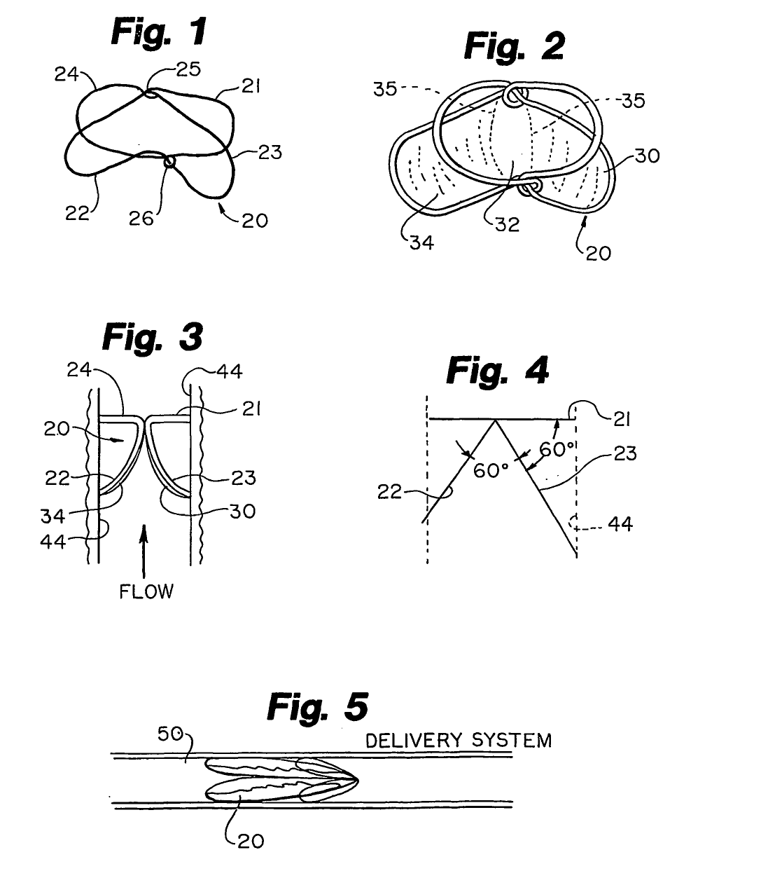

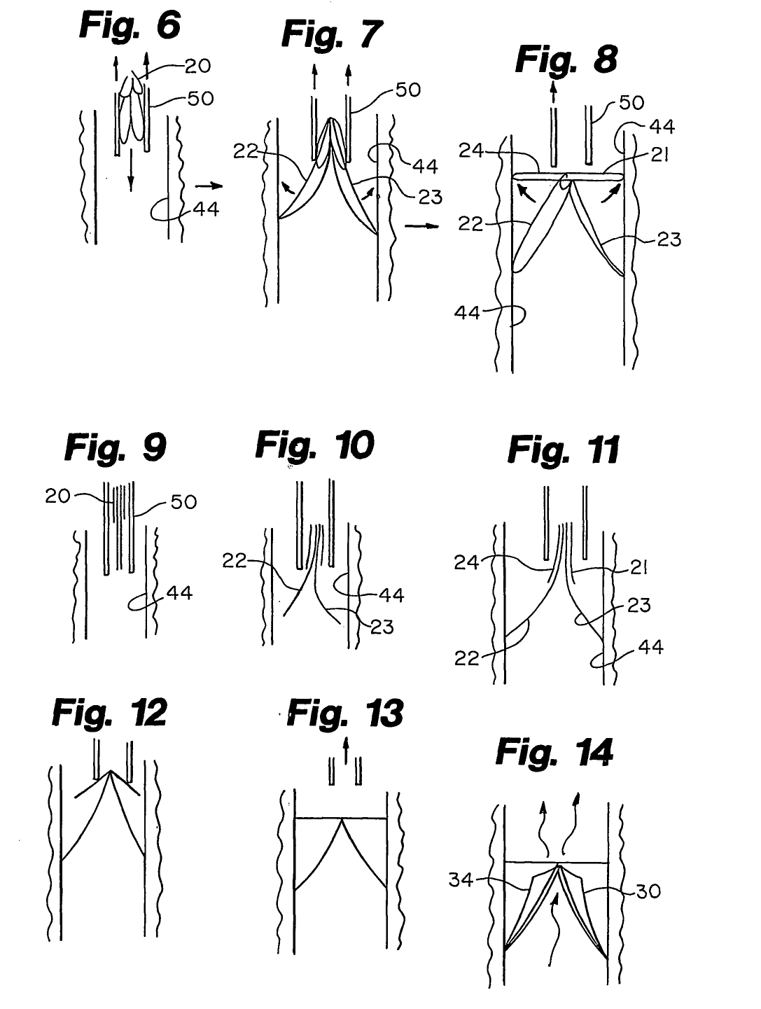

FIG. 1 is a schematic depiction of one embodiment of venous valve assembly 20 with a frame having a first support wing 21, an opposite second support wing 24, a first valve wing 22 with its opposite second valve wing 23. The first interlink 25 joins the support wings 21, 24 with the valve wings 22, 23 at a first junction. A second interlink 26 joins the support wings 21, 24 with the valve wings 22, 23 at a second junction. Valve 20 is preferably of unitary, single wire construction, but alternate configurations having a plurality of wires are possible.

FIG. 2 shows a venous valve assembly 20 with a first valve leaflet or flexible sheet 30 and a second valve leaflet or flexible sheet 34 with aperture 32 between the flexible sheets. It is recognized that, in operation, aperture 32 includes trailing edge portions 35 which open and closes as valve leaflets respond to the pressure and pumping action of the blood through the valve. As shown in FIG. 3, first support wing 21 and second support wing 24 provide lateral stability by exertion of outward radial force in the form of a support ring exerting outward pressure against the inner lumenal wall 44 at a venous location for the valve. In similar manner, the valve wings 22, 23 exert similar force in the form of a valve ring force exerted outwardly against the lumenal wall 44, and provide similar stabilizing and self-righting advantage to the valve as will be further discussed.

FIG. 4 illustrates the approximate included angle desired between each support wing 21 and each valve wing 23 as generally about 60°+/− about no more than 10° and preferably only about +/− a maximum of about 5°, and between first valve wing 23 and second valve wing 22 as their ends push into vein wall 44.

FIG. 5 illustrates the folding of the venous valve stent 20 to a closed position within a deployment system device 50. It is shown how the respective valve and support wings fold compactly together in an overlapping, butterfly-like relationship.

FIG. 6 illustrates the folded venous valve stent 20 inside a delivery system device 50, such as a catheter. FIGS. 7 and 8 further illustrate the deployment sequence of the replacement valve stent 20 in relation to a vein wall 44. The venous valve 20 is pushed toward the delivering end of the delivery system 50 until the first valve wing 23 and the second valve wing 22 spring open and engage the vein lumenal wall 44. The delivery system 50 is withdrawn after the venous valve wings are in the desired position. With the delivery system 50 separated from the venous valve stent 20, the first support wing 21 and the second support wing 24 then engage the vein wall 44.

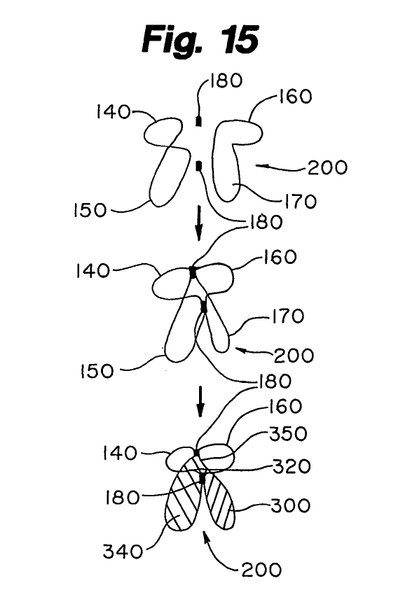

FIGS. 9-14 are simpler schematic depictions of the steps of delivering the venous valve stent 20 into a vein. The final step illustrates the position of the venous valve stent in relation to blood flow arrows and depicts the functionality of the valve leaflets.

FIG. 15 is an assembly sequence view of another embodiment of a venous valve assembly 200 in which the first support wing 140 is conjoined with the first valve wing 150 to form half of venous valve assembly 200. The second support wing 160 is conjoined with second valve wing 170 to form the other half of venous valve assembly 200. The two halves are attached by connectors 180 at opposite locations on the frame. The last sequence view in this figure shows the connected halves with first valve leaflet or flexible sheet 300 and second valve leaflet or flexible sheet 340 attached to the valve wings thereby forming aperture 320 with trailing edges 350 in operation.

Because numerous modifications may be made of this invention without departing from the spirit thereof, the scope of the invention is not to be limited to the embodiments illustrated and described. Rather, the scope of the invention is to be determined by the following claims and their equivalents.

1. A valve assembly, comprising:
a first valve wing coupled to a first support wing;
a second valve wing coupled to a second support wing;
a first flexible sheet fastened to the first valve wing; and
a second flexible sheet fastened to the second valve wing.

2. The valve assembly of claim 1, wherein:
the first flexible sheet and the second flexible sheet have a first position in which a trailing portion of the first flexible sheet contacts a trailing portion of the second flexible sheet for preventing blood flow therepast; and
the first flexible sheet and the second flexible sheet have a second position in which the trailing portion of the first flexible sheet and trailing portion of the second flexible sheet define an aperture for allowing blood flow through the valve.

3. The valve assembly of claim 2, wherein the first flexible sheet and the second flexible sheet are biased to assume the first position.

4. The valve assembly of claim 1, wherein the first valve wing, the second valve wing, the first support wing, and the second support wing are biased to assume an expanded shape.

5. The valve assembly of claim 1, wherein the first valve wing, the second valve wing, the first support wing, and the second support wing define a generally cylindrical surface when they assume the expanded shape.

6. The valve assembly of claim 1, wherein the first valve wing, the second valve wing, the first support wing, and the second support wing each comprise a length of wire having a U-shaped bend.

7. The valve assembly of claim 1, wherein the first valve wing, the second valve wing, the first support wing, and the second support wing are all formed from a single wire.

8. The valve assembly of claim 1, wherein a first end of the first valve wing is coupled to a first end of the first support wing by a first loop.

9. The valve assembly of claim 1, wherein a second end of the first valve wing is coupled to a second end of the second valve wing by a second loop.

10. The valve assembly of claim 1, wherein a first end of the second valve wing is coupled to a first end of the second support wing by a third loop.

11. The valve assembly of claim 1, wherein a second end of the second support wing is coupled to a second end of the first support wing by a fourth loop.

12. The valve assembly of claim 1, wherein:
a first end of the first valve wing is coupled to a first end of the first support wing by a first loop;
a second end of the first valve wing is coupled to a second end of the second valve wing by a second loop;
a first end of the second valve wing is coupled to a first end of the second support wing by a third loop;
the second support wing is coupled to a second end of the first support wing by a fourth loop;
the first loop and the third loop are interlinked; and
the second loop and the fourth loop are interlinked.

13. The valve assembly of claim 1, wherein;
a first end of the first valve wing is coupled to a first end of the first support wing by a first bend;
a second end of the first valve wing is coupled to a second end of the first support wing by a second bend;
a first end of the second valve wing is coupled to a first end of the second support wing by a third bend;
a second end of the second valve wing is coupled to a second end of the second support wing by a fourth bend; and
the first valve wing is coupled to the second valve wing by at least one coupling member.

14. The valve assembly of claim 1, wherein the first valve wing, the second valve wing, the first support wing, and the second support wing are biased to assume an expanded shape.

15. The valve assembly of claim 14, wherein the first valve wing, the second valve wing, the first support wing, and the second support wing define a generally cylindrical surface when they assume the expanded shape.

16. The valve assembly of claim 14, wherein the first valve wing and the second support wing define an first angle when the valve assembly assumes the expanded shape.

17. The valve assembly of claim 16 wherein the first angle is about a 60 degree angle.

18. The valve assembly of claim 14, wherein the second valve wing and the first support wing define a second angle when the valve assembly assumes the expanded shape.

19. The valve assembly of claim 16, wherein the second angle is about a 60 degree angle.

20. The valve assembly of claim 1, wherein the first valve wing and the second valve wing define an third angle when the valve assembly assumes the expanded shape.

21. The valve assembly of claim 18, wherein the third angle is about a 60 degree angle.

22. The valve assembly of claim 1, wherein:
the valve assembly has a length A and a radius B;
the first flexible sheet is coupled to the first valve wing at a plurality of fixing points and the location of each fixing point may be defined by a polar coordinate values including a value Z corresponding to a z-axis, value R corresponding to an r-axis, and a value theta corresponding to an angle theta;
a first fixing point of the first flexible sheet being located generally at theta=0, Z=0, and R=B;
a second fixing point of the first flexible sheet being located generally at theta=180, Z=0, and R=B; and
a third fixing point of the first flexible sheet being located generally at theta=90 degrees, Z=A, and R=B.

23. The valve assembly of claim 1, wherein:
the valve assembly has a length A and a radius B;
the second flexible sheet is coupled to the first valve wing at a plurality of fixing points and the location of each fixing point may be defined by a polar coordinate values including a value Z corresponding to a z-axis, value R corresponding to an r-axis, and a value theta corresponding to an angle theta;
a first fixing point of the second flexible sheet being located generally at theta=0, Z=0, and R=B;
a second fixing point of the second flexible sheet being located generally at theta=180, Z=0, and R=B; and
a third fixing point of the second flexible sheet being located generally at theta=270 degrees, Z=A, and R=B.

24. The valve assembly of claim 1, wherein the first flexible sheet is coupled to the first valve wing by a plurality of sutures.

25. The valve assembly of claim 1, wherein the first flexible sheet is coupled to the first valve wing by a staple.

26. The valve assembly of claim 1, wherein the first flexible sheet is coupled to the first valve wing by a hook.

27. The valve assembly of claim 1, wherein at least a portion of one of the flexible sheets includes either sclera or small intestine sub-mucosa material.

28. The valve assembly of claim 1, wherein the first valve wing, the second valve wing, the first support wing, and the second support wing comprise a resilient metallic material.

29. The valve assembly of claim 28, in which the resilient metallic material is selected from either nitinol or stainless steel.

30. The valve assembly of claim 1, wherein at least a portion of one of the flexible sheets includes mammalian tissue.

31. The valve assembly of claim 1, wherein the first valve wing and the first support wing are formed from a first wire, the second valve wing and the second support wing are formed from a second wire and the two parts are joined by a plurality of connectors.

32. The valve assembly of claim 1 where the expanded shape has a circumference larger than the circumference of the selected vascular lumen thereby inhibiting the assembly's movement from its desired position.

33. A method of using the valve assembly comprising the following steps:
a. inserting a delivery system containing the valve assembly into the vascular system;
b. deploying at least one replacement valve assembly at a selected site within a vein with a first valve wing pair and then a second support wing pair.
